(19) United States
(12) Patent Application Publication
(10) Pub. No.: US 2022/0117506 A1
(43) Pub. Date: Apr. 21, 2022

(54) ELECTROMYOGRAPHY SIGNAL ANALYSIS DEVICE AND ELECTROMYOGRAPHY SIGNAL ANALYSIS METHOD

(71) Applicant: Institute For Information Industry, Taipei (TW)

(72) Inventors: Yan-Tong LIU, Taipei (TW); Chun CHEN, Taipei (TW); Chih-Hao HSU, Taipei (TW)

(21) Appl. No.: 17/099,753

(22) Filed: Nov. 16, 2020

(30) Foreign Application Priority Data
Oct. 21, 2020 (TW) 109136523

(51) Int. Cl. A61B 5/04; A61B 5/0492; A61B 5/00

(52) U.S. Cl. CPC A61B 5/04012; A61B 5/7203; A61B 5/0492

(57) ABSTRACT

An electromyography signal analysis device and an electromyography signal analysis method are disclosed. The electromyography signal analysis device selects at least one reference indicator from a reference indicator set according to a category-selecting command from a user, and then determines at least one category of noise according to the at least one reference indicator. Next, the electromyography signal analysis device selects at least one filter from a filter set according to the at least one category of noise, and then configures the at least one filter. Subsequently, the electromyography signal analysis device filters an electromyography signal with the at least one configured filter, and analyzes the filtered electromyography signal according to the at least one reference indicator to generate an analysis result in response to the category-selecting command.

### PRIORITY

This application claims priority to Taiwan Patent Application No. 109136523 filed on Oct. 21, 2020, which is incorporated herein by reference in its entirety.

### FIELD

The present disclosure relates to a signal analysis device and a signal analysis method. More specifically, the present disclosure relates to an electromyography signal analysis device and an electromyography signal analysis method.

### BACKGROUND

An electromyography (EMG) signal is a signal generated by the potential difference between two sides of a muscle when the muscle contracts. The EMG signal may be used to present the active state of the muscle, and may be used for different categories of analysis (e.g., muscle fatigue detection, muscle lesion detection, or neural lesion detection) after being processed and quantified.

For different categories of analysis, the methods of signal processing and analysis are different, so it is usually necessary to choose different filters and adjust specific filter parameters in order to effectively eliminate the noise in the electromyography signal and carry out a subsequent analysis. However, the existing electromyography signal analysis devices are only designed for a single analysis category, and their respective filters are configured with unchangeable default parameters. In other words, the existing electromyography signal analysis device cannot dynamically change the type of their respective filters and adaptively adjust the filter parameters in response to different categories of analysis.

Therefore, when a user applies an existing electromyography signal analysis device suitable for a certain category of analysis to other categories of analysis (for example, using an electromyography signal analysis device suitable for detecting muscle fatigue to detect neural lesion), the electromyography signal analysis device is likely to filter out valuable information, or is unable to effectively filter out unnecessary noise, which all make subsequent analysis result inaccurate. Accordingly, an urgent need exists in the art to design an adaptive electromyography signal analysis device which may be applied to multiple categories of analysis.

### SUMMARY

To solve at least the above problems, the present invention provides in certain embodiments an electromyography signal analysis device. The electromyography signal analysis device may comprise a transceiver, a storage, and a processor electrically connected with the transceiver and the storage. The transceiver may be configured to receive an electromyography signal and a category-selecting command from a user. The storage may be configured to store a reference indicator set and a filter set. The processor may be configured to: select at least one reference indicator from the reference indicator set according to the category-selecting command; determine at least one category of noise according to the at least one reference indicator; select at least one filter from the filter set according to the at least one category of noise, and configure the at least one filter; filter the electromyography signal with the at least one configured filter, and analyze the filtered electromyography signal according to the at least one reference indicator to generate an analysis result in response to the category-selecting command.

To solve at least the above problems, the present invention further provides in certain embodiments an electromyography signal analysis method. The electromyography signal analysis method may be implemented on an electromyography signal analysis device, and the electromyography signal analysis device may store a reference indicator set and a filter set. The electromyography signal analysis method may comprise:

receiving, by the electromyography signal analysis device, an electromyography signal;

receiving, by the electromyography signal analysis device, a category-selecting command from a user;

selecting, by the electromyography signal analysis device, at least one reference indicator from the reference indicator set according to the category-selecting command;

determining, by the electromyography signal analysis device, at least one category of noise according to the at least one reference indicator;

selecting, by the electromyography signal analysis device, at least one filter from the filter set according to the at least one category of noise, and configuring the at least one filter;

filtering, by the electromyography signal analysis device, the electromyography signal with the at least one configured filter; and

analyzing, by the electromyography signal analysis device, the filtered electromyography signal according to the at least one reference indicator to generate an analysis result according to the category-selecting command.

The electromyography signal analysis device and the electromyography signal analysis method as provided in certain embodiments may select an appropriate filter and adjust parameters of the filter appropriately according to different category-selecting commands from users, and then correctly and effectively filter out the noise existing in the electromyography signal through the filter. Through such a filtering mechanism, the electromyography signal analysis device and method may always retain the valuable information and eliminate unnecessary noise, so they can adapt to various categories of analysis and produce appropriate and accurate analysis results. Accordingly, the electromyography signal analysis device and the electromyography signal analysis method as provided indeed solve the above problems.

This summary overall describes the core concept of the present invention, and covers the problem to be solved, the means to solve the problem, and the effect of the present invention to provide a basic understanding of the present invention by those of ordinary skill in the art. However, it shall be appreciated that, this summary is not intended to encompass all embodiments of the present invention but is provided only to present the core concept of the present invention in a simple form and as an introduction to the following detailed description.

### BRIEF DESCRIPTION OF THE DRAWINGS

The drawings can assist the description of the present disclosure, wherein:

FIG. 1 illustrates a schematic architectural view of an electromyography signal analysis device according to some embodiments of the present invention;

FIG. 2A illustrates a schematic view of the operation of the electromyography signal analysis device in FIG. 1;

FIG. 2B illustrates a schematic view regarding details of configuring the filter in FIG. 2A; and

FIG. 3 illustrates a schematic view of an electromyography signal analysis method according to some embodiments of the present invention.

### DETAIL DESCRIPTION

Example embodiments of the present invention described below are not intended to limit the present invention to any environment, applications, structures, processes or steps described in these example embodiments. In the attached drawings, elements unrelated to the present invention are omitted from depiction; and dimensions of elements and proportional relationships among individual elements in the attached drawings are only exemplary examples but not intended to limit the present invention. Unless stated particularly, same (or similar) element symbols may correspond to same (or similar) elements in the following description. Unless otherwise specified, “one” means a kind (category), not one (quantity). For example, a device means a kind of device, and does not mean one device. Unless otherwise specified, the number of the element is not limited.

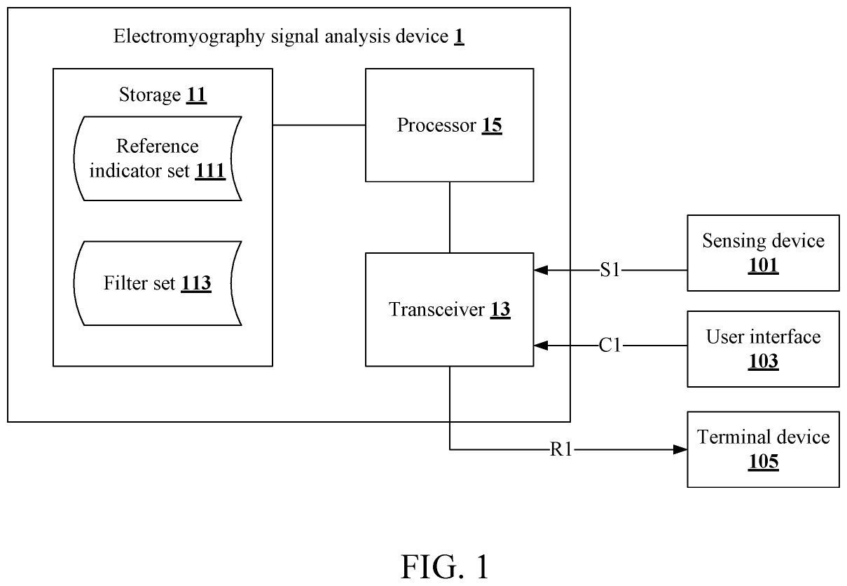

FIG. 1 illustrates a schematic architectural view of an electromyography signal analysis device according to some embodiments of the present invention. The content shown in FIG. 1 is for illustrating the embodiment of the present invention instead of limiting the scope claimed in the present invention.

Referring to FIG. 1, an electromyography signal analysis device 1 may basically comprise a storage 11, a transceiver 13, and a processor 15 which are electrically connected with each other. The electrical connection among the storage 11, the transceiver 13, and the processor 15 may be direct (i.e., connected not via other functional elements) or indirect (i.e., connected via other functional elements). The electromyography signal analysis device 1 may be one of the various types of computing devices, such as desktop computers, portable computers, smart phones, portable electronic accessories (glasses, watches, etc.), cloud servers or the like.

The storage 11 may be configured to store data generated by the electromyography signal analysis device 1, data transmitted to the electromyography signal analysis device 1 from external devices, or data input to the electromyography signal analysis device 1 by users themselves. The storage 11 may comprise a first-level memory (also referred to as main memory or internal memory), and the processor 15 may directly read instruction sets stored in the first-level memory, and execute these instruction sets if needed. In some embodiments, the storage 11 may further comprise a second-level memory (also referred to as external memory or auxiliary memory) in addition to the first-level memory, and the memory at this level may use a data buffer to transmit data stored to the first-level memory. For example, the second-level memory may for example be a hard disk, an optical disk or the like, without being limited thereto. In some embodiments, in addition to the first-level memory, the storage 11 may further comprise a third-level memory, i.e., a storage device that can be inserted into or pulled out from a computer directly, e.g., a mobile disk. In some embodiments, the storage 11 may also comprise a cloud storage.

The storage 11 may be configured to store a reference indicator set 111 and a filter set 113. The reference indicator set 111 may comprise multiple reference indicators, and different reference indicators can indicate the muscle state presented in the electromyography signal S1 in different aspects. In some embodiments, the reference indicator set 111 may be further divided into a time-domain reference indicator set and a frequency-domain reference indicator set according to different analysis methods. The time-domain reference indicator set may comprise multiple time-domain reference indicators used for time-domain analysis of the electromyography signal, such as but not limited to, a root mean square amplitude, an amplitude difference, an integrated electromyography, or a phase crossover order. The frequency-domain reference indicator set may comprise multiple frequency-domain reference indicators used for frequency-domain analysis of the electromyography signal, such as but not limited to, an average power frequency, a median frequency shift, a decreasing slope of an amplitude, or an amplitude threshold detection. The filter set 113 may comprise various types of filters, such as but not limited to, a Butterworth filter, a Hamming window filter, or a full-wave rectification filter. The basic function of these filters is to avoid or reduce noise generated for the electromyography signal S1 due to shaking of wires, actions of personnel, or radio waves.

The transceiver 13 may be configured to perform wired or wireless communication with a sensing device 101 to receive an electromyography signal S1 from the sensing device 101. The sensing device 101 may basically comprise a sensor and a transmission interface. The sensor may be an invasive sensor or a non-invasive sensor. The non-invasive sensor may comprise one or more electrode patches, which may be attached to the skin surface or placed on clothes, so as to measure a non-invasive surface electromyography (sEMG) signal. In the embodiment of disposing the electrode patches on clothes, one side of the electrode patch is disposed on the clothes, and the other side may contact the skin surface of the user when the clothes is worn by the user. The invasive sensor may comprise one or more needle electrodes which may be inserted into the muscle to measure an invasive electromyography signal. The transmission interface is configured to transmit the non-invasive surface electromyography signal or the invasive electromyography signal to the transceiver 13. In some embodiments, the sensing device 101 additionally comprises a control chip which is used to control the measurement and transmission of the sensing device 101.

In some embodiments, the transceiver 13 may also be configured to perform wired or wireless communication with a user interface 103 to receive a category-selecting command C1 of a user from the user interface 103. The category-selecting command C1 may be configured to instruct the electromyography signal analysis device 1 to adopt a signal processing and analysis mode corresponding to a certain category of analysis, and the category of analysis may be, for example but not limited to, muscle fatigue, muscle lesion, neural lesion, fitness application, or other application items. The user interface 103 may be independent of the electromyography signal analysis device 1, or may be directly disposed in the electromyography signal analysis device 1.

In some embodiments, the transceiver 13 may also be configured to send the analysis result R1 to the terminal device 105 in a wired or wireless manner, and the analysis result R1 may be a result of determination related to a certain category of analysis. The terminal device 105 may be a desktop computer, a portable computer, a smart phone, a portable electronic accessory (glasses, watch, etc.) or the like.

In some embodiments, the transceiver 13 may comprise a transmitter and a receiver. Taking wireless communication as an example, the transceiver 13 may comprise, but is not limited to, an antenna, an amplifier, a modulator, a demodulator, a detector, an analog-to-digital converter, a digital-to-analog converter, or other communication components. Taking wired communication as an example, the transceiver 13 may be, for example but not limited to, a gigabit Ethernet transceiver, a gigabit interface converter (GBIC), a small form-factor pluggable (SFP) transceiver, a ten gigabit small form-factor pluggable (XFP) transceiver or the like.

The processor 15 may include various microprocessors or microcontrollers capable of signal processing. A microprocessor or a microcontroller is a programmable specific integrated circuit that has the function of operation, storage, output/input or the like. Moreover, the microprocessor or the microcontroller can receive and process various coded instructions, thereby performing various logical operations and arithmetical operations and outputting corresponding operation results. The processor 15 may be programmed to interpret various instructions so as to analyze data in the electromyography signal analysis device 1 and execute various procedures or programs.

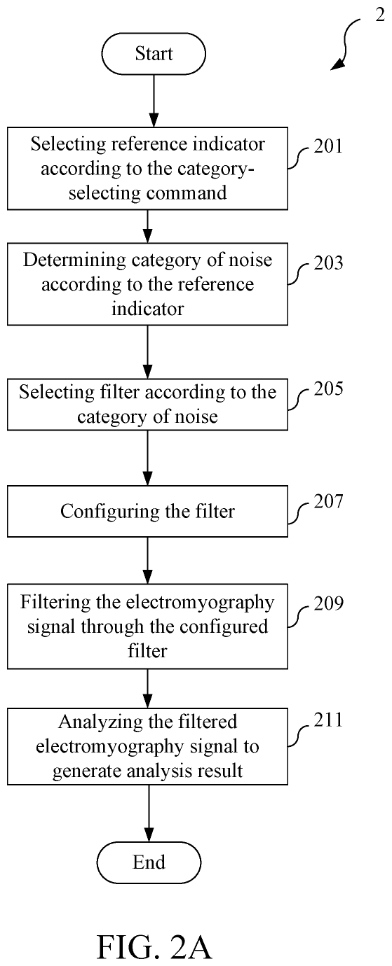

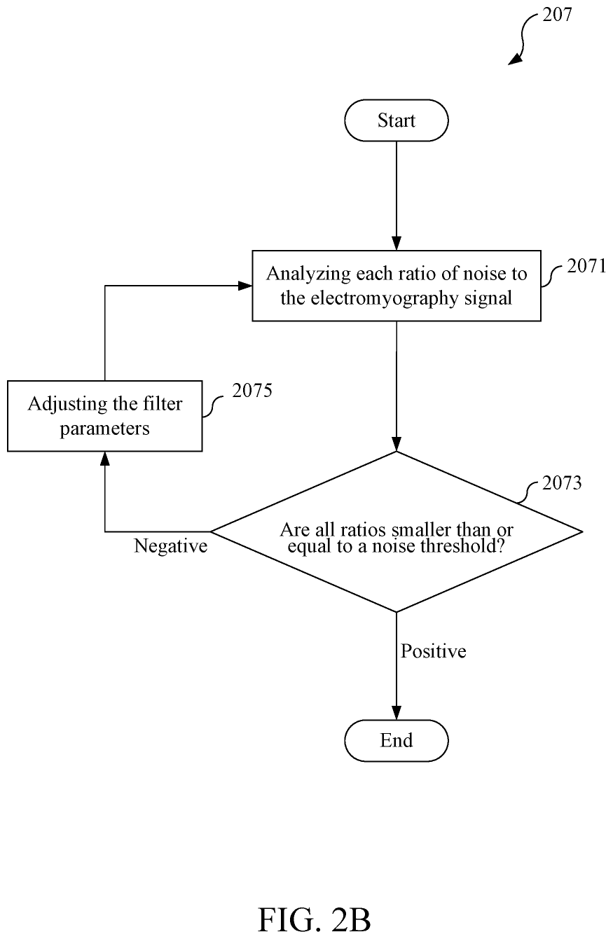

Next, how the electromyography signal analysis device 1 analyzes the electromyography signal S1 will be explained with reference to FIG. 2A and FIG. 2B. FIG. 2A illustrates a schematic view of the operation of an electromyography signal analysis device according to some embodiments of the present invention, and FIG. 2B illustrates a schematic view regarding details of configuring the filter in FIG. 2A. The contents shown in FIG. 2A and FIG. 2B are for illustrating the embodiments of the present invention instead of limiting the scope claimed in the present invention.

Referring to FIG. 1 and FIG. 2A together, the processor 15 may first select one or more reference indicators from the reference indicator set 111 based on the category-selecting command C1 from a user (labeled as action 201). Further speaking, the processor 15 may analyze the electromyography signal S1 by using a specific reference indicator to obtain the main features in the electromyography signal S1 that are related to the category of analysis indicated by the category-selecting command C1. In some embodiments, methods for analyzing an electromyography signal may comprise time-domain analysis and frequency-domain analysis. The time-domain analysis is to show how the amplitude of signal changes in time dimension with the root mean square value, the average amplitude, or the integrated electromyography of the electromyography signal. The frequency-domain analysis is to analyze the frequency spectrum obtained after performing fast Fourier transformation (FFT) on the electromyography signal, and show the frequency characteristics of the electromyography signal with indicators such as the average power frequency, the median frequency or the like. Accordingly, the reference indicator set 111 may be further divided into a time-domain reference indicator set and a frequency-domain reference indicator set according to the above two analysis methods.

As mentioned above, the processor 15 selects appropriate reference indicators from the time-domain reference indicator set and the frequency-domain reference indicator set respectively according to the category-selecting command C1, so as to evaluate the electromyography signal S1 correctly. For example, when the category-selecting command C1 indicates “Muscle fatigue”, the processor 15 may select “Amplitude difference” from the time-domain reference indicator set and select “Median frequency shift” from the frequency-domain reference indicator set as the indicators for evaluation. The amplitude difference indicates the peak-to-peak value of the electromyography signal, which may be used to evaluate the load intensity of the muscle. The larger the force that the muscle exerts is, the greater the amplitude difference of the electromyography signal will be. If the amplitude difference decreases gradually, then it means that the strength of the force exerted by the muscle decreases gradually. When the amplitude difference is lower than a preset threshold, it means that muscle fatigue occurs. The median frequency may be used to indicate the frequency distribution of the electromyography signal. If the median frequency continues to decrease until it is lower than a preset threshold, it represents a phenomenon of muscle fatigue. It shall be noted that, the number and types of the above-mentioned reference indicators are only examples and are not limited. How to select relevant reference indicators and use these reference indicators to analyze the electromyography signal and evaluate the muscle state shall be well known by those of ordinary skill in the art, and thus will not be further described herein. Each of the preset threshold of the amplitude difference and the median frequency may be set by the processor 15 according to historical data, a user's demand, the type of the sensing device 101, or a combination of the above conditions. It shall be noted that, the above-mentioned methods of setting the preset threshold are only an example and are not limited.

In some embodiments, before analyzing the electromyography signal S1, the processor 15 may pre-process the electromyography signal S1. In detail, the electromyography signal S1 may be interfered by various noises during transmission (e.g., radiation interference during wire transmission, noise caused by body shaking, moving artifact between the electrode and the skin, etc.) so that erroneous features not belonging to the original electromyography signal (i.e., the electromyography signal not interfered by the noise) are captured during the process of analyzing the electromyography signal S1, thereby causing inaccurate analysis results. Therefore, before analyzing the electromyography signal S1, preliminary noise filtering may be performed on the signal to improve the accuracy of subsequent analysis. For example, before starting the analysis, the processor 15 may first input the electromyography signal S1 to a differential amplifier so that the common-mode part (i.e., common-mode noise) in the electromyography signal S1 is eliminated by subtracting the positive and negative terminals of the differential amplifier, while the differential-mode part (i.e., the electromyography signal other than the common-mode noise) is amplified based on an amplification factor of the differential amplifier. In this way, a signal with less distortion can be obtained, thereby improving the accuracy of subsequent signal analysis. For another example, the processor 15 may also input the electromyography signal S1 to a band-pass filter to filter out signals in frequencies outside the passband defined by a lower cut-off frequency and an upper cut-off frequency, so as to retain the most representative signals.

In view of the fact that the aforesaid pre-processing performed on the electromyography signal S1 cannot completely eliminate all noises, the processor needs to perform a further noise filtering operation on the electromyography signal S1. Because the signal features captured according to each reference indicator are different, the categories of noise that are influential in the process of being captured are also different. Therefore, after the completion of the action 201, the processor 15 may determine at least one category of noise corresponding to the at least one selected reference indicator according to the at least one selected reference indicator (labeled as action 203), thereby filtering out more influential noise. The category of noise may include, for example but not limited to, noise from spectral leakage, aliasing noise, natural noise, oscillation noise, noise from ripple effect, and noise from inductive effect. For example, when “Amplitude difference” and “Median frequency shift” are selected as the reference indicators, the processor 15 may decide to take “Noise from spectral leakage”, “Noise from ripple effect”, “Noise from inductive effect”, “Aliasing noise” and “Natural noise” as the main categories of noise to be filtered without considering the influence caused by “Oscillating noise”.

After the completion of the action 203, the processor 15 may select, based on one or more categories of noise determined, one or more filters from the filter set 113 that are suitable for filtering the noise corresponding to the one or more categories of noise (labeled as action 205), and then configure the one or more selected filters (labeled as action 207). In some embodiments, the processor 15 configures the selected filter(s) in the way as shown in FIG. 2B. Specifically, the processor 15 may analyze each ratio of noise corresponding to the determined category(s) of noise to the electromyography signal S1 (labeled as action 2071) (herein, the processor 15 compares the average amplitude of the electromyography signal S1 with the average amplitude of noise of each category to obtain the respective ratios of noise to the electromyography signal S1, so the sum of the ratios is not necessarily 100%), then the processor 15 may determine whether each ratio of all the ratios is smaller than a noise threshold (labeled as action 2073). If the above result of the determination is “Negative”, then the processor 15 may adjust the filter parameters of the selected filter(s) in a way suitable for the categories of noise (labeled as action 2075), so as to reduce the ratios of noise corresponding to the determined category(s) of noise. Filter parameters may be, but are not limited to, an order, a constant, or a frequency of a filter. For example, in order to reduce the ratio of the noise from spectral leakage to the electromyography signal S1, the processor 15 may increase the order of a Butterworth filter. The above method of adjusting filter parameters is only an example instead of a limitation. Therefore, in addition to the method of adjusting filter parameters described above, other methods of adjusting filter parameters known and feasible in the art can also be adopted herein.

After the completion of the action 2075, the processor 15 returns to the action 2071 to re-analyze each ratio of noise corresponding to the determined category(s) of noise to the electromyography signal S1. If any of the ratios of noise to the electromyography signal S1 is not smaller than its noise threshold, then the processor 15 will go back to the action 2075 and adjust the filter parameters again. Therefore, the processor 15 may adjust the filter parameters of the selected filter(s) to appropriate values by repeating the steps 2071, 2073, and 2075. The noise threshold corresponding to each category of noise may be set by users, and may be changed to other values according to the users' demand for the accuracy of signal analysis. When the required accuracy is higher, the noise threshold corresponding to each of all categories of noise should be set smaller (for example, less than 3%, or 1%). In contrast, when the required accuracy is lower, the noise threshold corresponding to each of all categories of noise may be set larger (for example, less than 20%, 15%, or 10%). In some embodiments, the noise thresholds corresponding to different categories of noise may be set differently according to the category-selecting command of the user.

In some embodiments, the processor 15 may configure the filter through a support vector machine model. The support vector machine model may be pre-established by the processor 15 according to a support vector machine algorithm and stored in the storage 11. Alternatively, the support vector machine model may also be pre-established by other external devices and stored in the storage 11 in advance. With the support vector machine model, the processor 15 may repeatedly analyze the ratio of noise corresponding to each category to the electromyography signal S1 and repeatedly adjust the filter parameters as shown in FIG. 2B.

Returning to FIG. 2A, after the completion of the action 207 (i.e., after the configuration of the filter(s) is completed), the processor 15 may filter the electromyography signal S1 through the filter(s) configured by the action 207 (labeled as action 209), and then analyze the filtered electromyography signal S1 to generate analysis result R1 in response to the category-selecting command C1 (labeled as action 211). Specifically, the processor 15 analyzes the electromyography signal S1 according to the at least one reference indicator which is selected based on the category-selecting command C1, to generates the analysis result R1 in response to the category-selecting command C1. For example, when the category-selecting command C1 indicates “Muscle fatigue” as the category of analysis, the processor 15 may select two reference indicators, i.e., “Amplitude difference” and “Median frequency shift”, as the indicators for evaluating the muscle state, and analyze the filtered electromyography signal S1 according to these two reference indicators. Then, when the processor 15 determines that the “Amplitude difference” of the electromyography signal S1 is greater than “3” (that is, the ratio of the maximum amplitude to the minimum amplitude is more than “3”) and that the “Slope of median frequency shift” is greater than a constant “2” (that is, the ratio of the energy variation to the median frequency variation of the electromyography signal is more than 2), the processor 15 determines that the electromyography signal S1 represents a phenomenon of muscle fatigue and generates the analysis result R1 representing muscle fatigue. The above-mentioned “Amplitude difference is greater than ‘3’” is only an example, and the “Amplitude difference” may be set according to different required accuracy or user requirements. Also, the above-mentioned “Slope of median frequency shift is greater than ‘2’” is only an example, and the “Slope of median frequency shift” may be set according to different required accuracy or user requirements.

According to the above descriptions, the processor 15 can dynamically select the appropriate filter(s) and adjust the relevant filter parameters according to the category-selecting command C1 so that the configured filter can effectively filter out the specific noise related to the category-selecting command C1 from the electromyography signal S1. The processor 15 can also select the corresponding reference indicator according to the category-selecting command C1, so as to adaptively analyze the filtered electromyography signal S1 and generate accurate analysis result R1. Therefore, the electromyography signal analysis device 1 is an adaptive electromyography signal analysis device that can be adapted for different categories of analysis.

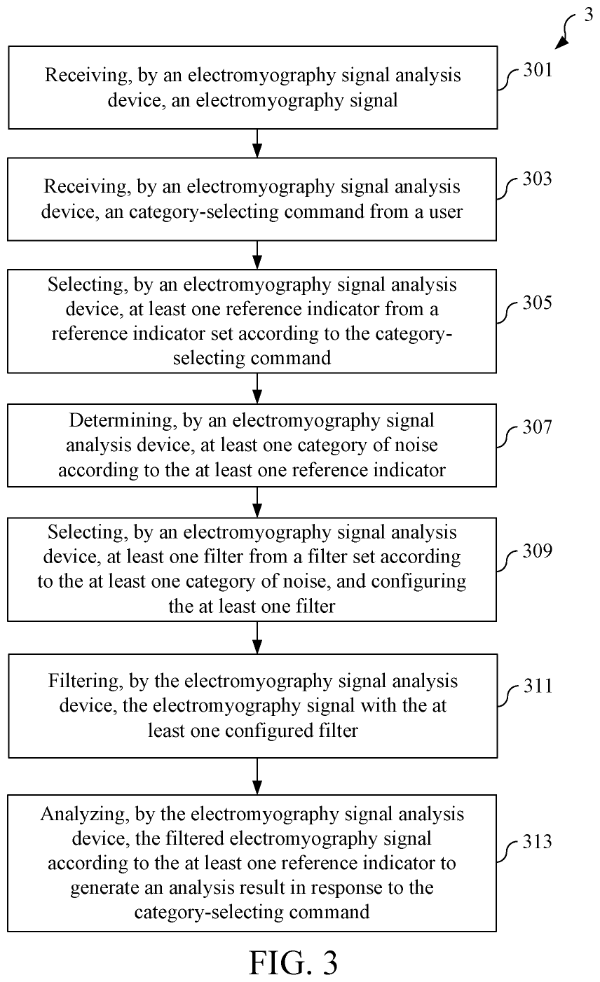

FIG. 3 illustrates a schematic view of an electromyography signal analysis method according to some embodiments of the present invention. The content shown in FIG. 3 is only for illustrating the embodiment of the present invention instead of limiting the scope claimed in the present invention.

Referring to FIG. 3, an electromyography signal analysis method 3 may be implemented on an electromyography signal analysis device. The electromyography signal analysis device may store a reference indicator set and a filter set, and the electromyography signal analysis method 3 may comprise the following steps:

receiving, by the electromyography signal analysis device, an electromyography signal (labeled as step 301);

receiving, by the electromyography signal analysis device, a category-selecting command from a user (labeled as step 303);

selecting, by the electromyography signal analysis device, at least one reference indicator from the reference indicator set according to the category-selecting command (labeled as step 305);

determining, by the electromyography signal analysis device, at least one category of noise according to the at least one reference indicator (labeled as step 307);

selecting, by the electromyography signal analysis device, at least one filter from the filter set according to the at least one category of noise, and configuring the at least one filter (labeled as step 309);

filtering, by the electromyography signal analysis device, the electromyography signal with the at least one configured filter (labeled as step 311); and

analyzing, by the electromyography signal analysis device, the filtered electromyography signal according to the at least one reference indicator to generate an analysis result in response to the category-selecting command (labeled as step 313).

The order of the steps 301 to 313 shown in FIG. 3 is not intended to limit the scope claimed in the present invention. The order of the steps 301 to 313 may be arbitrarily changed without affecting the implementation of the electromyography signal analysis method 3. For example, the step 301 may be performed earlier or later than the step 303. Optionally, the step 301 and the step 303 may also be executed simultaneously.

In some embodiments, the electromyography signal analysis method 3 may further comprise the following step:

Repeatedly adjusting filter parameters of the at least one filter by the electromyography signal analysis device until each ratio of noise corresponding to the at least one category of noise to the electromyography signal is less than a noise threshold, thereby generating the at least one configured filter.

In some embodiments, the electromyography signal analysis method 3 may further comprise the following step:

repeatedly adjusting filter parameters of the at least one filter by the electromyography signal analysis device with a support vector machine model until each ratio of noise corresponding to the at least one category of noise to the electromyography signal is less than a noise threshold, thereby generating the at least one configured filter.

In some embodiments, the electromyography signal analysis method 3 may further comprise the following step:

sending the analysis result to a terminal device by the electromyography signal analysis device.

In some embodiments, for the electromyography signal analysis method 3, the electromyography signal is an invasive electromyography signal or a non-invasive surface electromyography signal.

In some embodiments, for the electromyography signal analysis method 3, the at least one category of noise comprises at least one of noise from spectral leakage, aliasing noise, natural noise, oscillation noise, noise from ripple effect, and noise from inductive effect.

In some embodiments, for the electromyography signal analysis method 3, the filter set comprises at least two of a Butterworth filter, a Hamming window filter, and a full-wave rectification filter.

In some embodiments, for the electromyography signal analysis method 3, the reference indicator set comprises a time-domain reference indicator set and a frequency-domain reference indicator set.

In some embodiments, for the electromyography signal analysis method 3, the reference indicator set comprises a time-domain reference indicator set and a frequency-domain reference indicator set. Moreover, the time-domain reference indicator set comprises at least two of a root mean square amplitude, an amplitude difference, an integrated electromyography, and a phase crossover order, and the frequency-domain reference indicator set comprises at least two of an average power frequency, a median frequency shift, a decreasing slope of an amplitude, and an amplitude threshold detection.

Each embodiment of the electromyography signal analysis method 3 basically corresponds to a certain embodiment of the electromyography signal analysis device 1. Therefore, those of ordinary skill in the art can fully understand and implement all the corresponding embodiments of the electromyography signal analysis method 3 simply by referring to the above description for the electromyography signal analysis device 1, even though each embodiment of the electromyography signal analysis method 3 is not described in detail above.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. An electromyography signal analysis device, comprising:
a transceiver, being configured to receive an electromyography signal and a category-selecting command from a user;
a storage, being configured to store a reference indicator set and a filter set; and
a processor, being electrically connected with the transceiver and the storage, and being configured to:
select at least one reference indicator from the reference indicator set according to the category-selecting command;
determine at least one category of noise according to the at least one reference indicator;
select at least one filter from the filter set according to the at least one category of noise, and configure the at least one filter;
filter the electromyography signal with the at least one configured filter; and
analyze the filtered electromyography signal according to the at least one reference indicator to generate an analysis result in response to the category-selecting command.

2. The electromyography signal analysis device of claim 1, wherein the processor is further configured to: repeatedly adjust filter parameters of the at least one filter until each ratio of noise corresponding to the at least one category of noise to the electromyography signal is less than a noise threshold, thereby generating the at least one configured filter.

3. The electromyography signal analysis device of claim 2, wherein the processor repeatedly adjusts the filter parameters of the at least one filter through a support vector machine model.

4. The electromyography signal analysis device of claim 1, wherein the transceiver is further configured to send the analysis result to a terminal device.

5. The electromyography signal analysis device of claim 1, wherein the electromyography signal is an invasive electromyography signal or a non-invasive surface electromyography signal.

6. The electromyography signal analysis device of claim 1, wherein the at least one category of noise comprises at least one of noise from spectral leakage, aliasing noise, natural noise, oscillation noise, noise from ripple effect, and noise from inductive effect.

7. The electromyography signal analysis device of claim 1, wherein the filter set comprises at least two of a Butterworth filter, a Hamming window filter and a full-wave rectification filter.

8. The electromyography signal analysis device of claim 1, wherein the reference indicator set comprises a time-domain reference indicator set and a frequency-domain reference indicator set.

9. The electromyography signal analysis device of claim 8, wherein:
the time-domain reference indicator set comprises at least two of a root mean square amplitude, an amplitude difference, an integrated electromyography and a phase crossover order; and
the frequency-domain reference indicator set comprises at least two of an average power frequency, a median frequency shift, a decreasing slope of an amplitude and an amplitude threshold detection.

10. An electromyography signal analysis method implemented on an electromyography signal analysis device, the electromyography signal analysis device storing a reference indicator set and a filter set, the electromyography signal analysis method comprising:
receiving, by the electromyography signal analysis device, an electromyography signal;
receiving, by the electromyography signal analysis device, a category-selecting command from a user;
selecting, by the electromyography signal analysis device, at least one reference indicator from the reference indicator set according to the category-selecting command;
determining, by the electromyography signal analysis device, at least one category of noise according to the at least one reference indicator;
selecting, by the electromyography signal analysis device, at least one filter from the filter set according to the at least one category of noise, and configuring the at least one filter;
filtering, by the electromyography signal analysis device, the electromyography signal with the at least one configured filter; and
analyzing, by the electromyography signal analysis device, the filtered electromyography signal according to the at least one reference indicator to generate an analysis result in response to the category-selecting command.

11. The electromyography signal analysis method of claim 10, further comprising:
repeatedly adjusting filter parameters of the at least one filter by the electromyography signal analysis device until each ratio of noise corresponding to the at least one category of noise to the electromyography signal is less than a noise threshold, thereby generating the at least one configured filter.

12. The electromyography signal analysis method of claim 11, wherein a support vector machine model is used to repeatedly adjust the filter parameters of the at least one filter.

13. The electromyography signal analysis method of claim 10, further comprising:
sending the analysis result to a terminal device by the electromyography signal analysis device.

14. The electromyography signal analysis method of claim 10, wherein the electromyography signal is an invasive electromyography signal or a non-invasive surface electromyography signal.

15. The electromyography signal analysis method of claim 10, wherein the at least one category of noise comprises at least one of noise from spectral leakage, aliasing noise, natural noise, oscillation noise, noise from ripple effect and noise from inductive effect.

16. The electromyography signal analysis method of claim 10, wherein the filter set comprises at least two of a Butterworth filter, a Hamming window filter and a full-wave rectification filter.

17. The electromyography signal analysis method of claim 10, wherein the reference indicator set comprises a time-domain reference indicator set and a frequency-domain reference indicator set.

18. The electromyography signal analysis method of claim 17, wherein:
the time-domain reference indicator set comprises at least two of a root mean square amplitude, an amplitude difference, an integrated electromyography, and a phase crossover order; and
the frequency-domain reference indicator set comprises at least two of an average power frequency, a median frequency shift, a decreasing slope of an amplitude and an amplitude threshold detection.
